(19) United States
(12) Patent Application Publication
(10) Pub. No.: US 2015/0306164 A1
(43) Pub. Date: Oct. 29, 2015

(54) USES OF BACOPA MONNIERI EXTRACT

(71) Applicant: SOHO FLORDIS INTERNATIONAL PTY LTD, St Leonards, New South Wales (AU)

(72) Inventors: Andrew SCHOLEY, Hawthorn (AU); Con STOUGH, Hawthorn (AU)

(73) Assignee: SOHO FLORDIS INTERNATIONAL PTY LTD, St Leonards, New South Wales (AU)

(21) Appl. No.: 14/648,978

(22) PCT Filed: Dec. 3, 2013
(86) PCT No.: PCT/AU2013/001398

§ 371 Date: Jun. 2, 2015

(30) Foreign Application Priority Data
Dec. 3, 2012 (AU) 2012905250

(51) Int. Cl. A61K 36/68

(52) U.S. Cl. CPC A61K 36/68

(57) ABSTRACT

The present invention broadly relates to methods for acutely improving/enhancing cognitive performance in a human subject comprising administration of an extract of Bacopa monnieri.

### TECHNICAL FIELD

The present invention broadly relates to methods for acutely improving/enhancing cognitive performance in a human subject comprising administration of an extract of Bacopa monnieri.

### BACKGROUND OF THE INVENTION

Bacopa monnieri is a perennial creeping herb that inhabits wetlands and muddy shores. It has been used in traditional Ayurvedic medicine for its purported anti-amnesic, sedative, memory enhancing, anti-epileptic and anxiolytic effects for thousands of years.

The present inventors have surprisingly discovered that extracts of Bacopa monnieri acutely enhance cognitive performance in humans who are mentally stressed, mentally fatigued and/or cognitively challenged.

### SUMMARY OF THE INVENTION

In a first aspect the present invention provides a method for acutely improving/enhancing cognitive performance in a human subject, wherein the subject is mentally stressed, mentally fatigued and/or cognitively challenged, the method comprising administration to the subject of an extract of Bacopa monnieri.

The extract may be prepared from stems, leaves and roots of Bacopa monnieri.

The extract may be an alcoholic extract, and in one embodiment is an aqueous alcoholic extract, for example an aqueous C₁-C₆ alcoholic extract. In one embodiment, the aqueous C₁-C₆ alcoholic extract is a 50% (v/v) aqueous alcoholic extract. The C₁-C₆ alcohol may be ethanol.

The extract may comprise at least 55% (w/w) bacosides.

The extract may be administered to the subject prior to, during, or after the subject being mentally stressed, mentally fatigued and/or cognitively challenged. Typically, the extract is administered to the subject prior to the subject being mentally stressed, mentally fatigued and/or cognitively challenged. In one embodiment, the extract is administered at least or about 15 minutes prior to, at least or about 30 minutes prior to, at least or about 1 hour prior to, or at least or about 2 hours prior to, the subject being mentally stressed, mentally fatigued and/or cognitively challenged.

The subject may be mentally stressed, mentally fatigued and/or cognitively challenged as a result of undergoing a test, examination or some other activity involving cognition.

The extract may be administered prior to commencement of the test, examination or other activity involving cognition.

The extract may be administered at least or about 15 minutes prior to, at least or about 30 minutes prior to, at least or about 1 hour prior to, or at least or about 2 hours prior to, commencement of the test, examination or other activity involving cognition.

The extract may be administered in an amount between about 200 mg and about 2.0 g, or in an amount between about 300 mg and about 1.0 g, or in an amount between about 320 mg and about 960 mg, or in an amount between about 320 mg and about 640 mg.

The extract may be administered in an amount of at least about 320 mg, or in an amount of at least about 640 mg, or in an amount of at least about 960 mg.

In a second aspect the present invention provides use of an extract of Bacopa monnieri for acutely improving/enhancing cognitive performance in a human subject, wherein the subject is mentally stressed, mentally fatigued and/or cognitively challenged.

In a third aspect the present invention provides use of an extract of Bacopa monnieri in the manufacture of a medicament for acutely improving/enhancing cognitive performance in a human subject, wherein the subject is mentally stressed, mentally fatigued and/or cognitively challenged.

### BRIEF DESCRIPTION OF THE DRAWINGS

Embodiments of the invention are described herein with reference, by way of example only, to the following drawing.

FIG. 1: A screenshot from the Purple multi-tasking framework (MTF). Tasks include (clockwise from the top left): mental arithmetic, stroop, memory search, and visual tracking.

### DEFINITIONS

The following are some definitions that may be helpful in understanding the description of the present invention. These are intended as general definitions and should in no way limit the scope of the present invention to those terms alone, but are put forth for a better understanding of the following description.

Throughout this specification, unless the context requires otherwise, the word “comprise”, or variations such as “comprises” or “comprising”, will be understood to imply the inclusion of a stated step or element or integer or group of steps or elements or integers, but not the exclusion of any other step or element or integer or group of elements or integers. Thus, in the context of this specification, the term “comprising” means “including principally, but not necessarily solely”.

In the context of this specification, the terms “a” and “an” refer to one or to more than one (i.e. to at least one) of the grammatical object of the article. By way of example, “an element” means one element or more than one element.

In the context of this specification, the term “about” is understood to refer to a range of numbers that a person of skill in the art would consider equivalent to the recited value in the context of achieving the same function or result.

In the context of this specification, reference to a range of numbers disclosed herein (for example, 1 to 10) also incorporates reference to all rational numbers within that range (for example, 1, 1.1, 2, 3, 3.9, 4, 5, 6, 6.5, 7, 8, 9 and 10) and also any range of rational numbers within that range (for example, 2 to 8, 1.5 to 5.5 and 3.1 to 4.7) and, therefore, all sub-ranges of all ranges expressly disclosed herein are hereby expressly disclosed. These are only examples of what is specifically intended and all possible combinations of numerical values between the lowest value and the highest value enumerated are to be considered to be expressly stated in this application in a similar manner.

As used herein, the term “and/or” means “and” or “or” or both.

As used herein the term “extract” refers to an active preparation derived from Bacopa monnieri. In the context of the specification by “active” it is meant that the extract is capable of producing a desired effect as disclosed herein. An extract is obtained by a process of “extraction” which will be understood by those skilled in the art as, in general terms, treating plant material with a solvent, a liquid, or a supercritical fluid to dissolve the active preparation and separate the same from residual unwanted plant material. An extract may be in liquid form (for example as a decoction, solution, infusion or tincture) or solid form (for example as a powder or granules).

In the context of this specification, the term “acutely” is understood to mean a relatively rapid onset of a beneficial cognitive effect. An acute effect is distinct from a chronic effect which is an effect that occurs over a longer time period. Those skilled in the art readily understand and appreciate the difference between an acute effect and a chronic effect.

In the context of this specification, the term “improving/enhancing” as it relates to cognitive performance is understood to mean that cognitive performance is superior or better in a subject who is administered an extract of Bacopa monnieri when compared to cognitive performance of the subject in the absence of an extract of Bacopa monnieri. Improvement/enhancement may be assessed by comparing the cognitive performance of a subject who is administered an extract of Bacopa monnieri with the cognitive performance of the same subject in the absence of an extract of Bacopa monnieri. The improvement/enhancement may be qualitative or quantitative. The improvement/enhancement may be at least or about 5%, 10%, 15%, 20%, 25%, 30%, 35%, 40%, 45%, 50%, 55%, 60%, 65%, 70%, 75%, 80%, 85%, 90% or 95%.

In the context of this specification, the term “cognitive performance” is understood to mean the ability or capacity of a subject in carrying out a task that involves or requires cognition, such as for example thinking, reasoning, understanding, problem solving and/or decision making.

In the context of this specification, the term “cognitively challenged” is understood to mean that the subject is faced with a task or problem that involves or requires cognition, such as for example thinking, reasoning, understanding, problem solving and/or decision making, in particular a difficult or complex task or problem that may give rise to mental stress or mental fatigue in the subject.

In the context of this specification, the term “mentally stressed” is understood to mean strain or tension associated with thinking, reasoning, understanding, problem solving and/or decision making.

In the context of this specification the term “mentally fatigued” is understood to mean tiredness or exhaustion associated with thinking, reasoning, understanding, problem solving and/or decision making.

### DETAILED DESCRIPTION OF THE INVENTION

The present inventors have conducted a double-blind placebo controlled study examining the acute effects of two doses (320 mg and 640 mg) of an extract of Bacopa monnieri on participants' mood, cardiovascular activity and performance in a mentally effortful cognitive task (a cognitive demand battery) before and after administration of the extract (Example 1). The present inventors have also conducted a double-blind placebo controlled study to replicate and extend the findings of the initial study and to also assess different dosages of an extract of Bacopa monnieri (320 mg, 640 mg, and 960 mg) on cognitive performance and mood effects (Example 2).

Completion of the cognitive demand battery elicited the expected increase in participants' experience of feelings of stress and fatigue. No attenuation of either rating was observed for both doses in comparison to placebo. This result suggests that at the doses assessed Bacopa monnieri does not attenuate the experience of experimentally induced stress or fatigue after 1 hour of cognitively demanding assessment.

Assessment of Bacopa monnieri effect upon cardiovascular activity was undertaken utilising central and brachial assessments of blood pressure, and an assessment of arterial stiffness (augmentation index; derived from pulse pressure and augmentation pressure). No treatment related effects were observed in relation to blood pressure and arterial stiffness. Modulation of mood or brain activity elicited from consumption of Bacopa monnieri might be better assessed utilizing Electroencephalography (EEG) to monitor brain activity during the absorption phase to identify psychopharmacological effects that the high temporal resolution and relatively good spatial resolution that EEG produces to initially capture any acute effects of Bacopa monnieri on brain activity. Near infra-red spectroscopy could also be utilised to monitor cerebral blood flow and hemodynamics, via changes in oxygenated and deoxygenated haemoglobin in brain regions of interest during cognitive tasks.

Following completion of six repetitions of the cognitive demand battery, change from baseline scores indicated that the 320 mg extract improved performance at the first, second and fourth repetition post-dosing. The largest discrepancy in performance occurred at the fourth repetition, possibly indicating an optimal effect at a time when participants would be expected to be reaching the highest levels of mental stress and fatigue. Accordingly, the present invention is predicated on the finding by the inventors that extracts of Bacopa monnieri acutely enhance cognitive performance in humans who are mentally stressed, mentally fatigued and/or cognitively challenged.

In one aspect the present invention provides a method for acutely improving/enhancing cognitive performance in a human subject, wherein the subject is mentally stressed, mentally fatigued and/or cognitively challenged, the method comprising administration to the subject of an extract of Bacopa monnieri.

In another aspect the present invention provides use of an extract of Bacopa monnieri for acutely improving/enhancing cognitive performance in a human subject, wherein the subject is mentally stressed, mentally fatigued and/or cognitively challenged.

In a further aspect the present invention provides use of an extract of Bacopa monnieri in the manufacture of a medicament for acutely improving/enhancing cognitive performance in a human subject, wherein the subject is mentally stressed, mentally fatigued and/or cognitively challenged.

Extracts for use in accordance with the invention may be aqueous and/or organic solvent based extracts, obtained by single, combined and/or successive extraction of any available part of Bacopa monnieri, such as leaves, stems, roots, shoots, seeds and/or flowers. In one embodiment, the extract is obtained from leaves, roots and stems. Suitable extraction processes, and suitable solvents and liquids for extraction are known to those skilled in the art. Suitable solvents that may be used in solvent extraction methods include, but are not limited to water, alcohols, acetone, chlorinated solvents and ether solvents, such as diethyl ether and THF. In some embodiments the extract is an alcoholic extract, and in particular an aqueous alcoholic extract. Suitable alcohols will be well known to those skilled in the art. Typically, the alcohol is a C₁-C₆ alcohol, for example ethanol. In one embodiment, the extract is obtained by extraction of Bacopa monnieri with an aqueous alcoholic mixture comprising between about 10% (v/v) and about 90% (v/v) alcohol, or between about 20% (v/v) and about 80% (v/v) alcohol, or between about 30% (v/v) and about 70% (v/v) alcohol, or between about 40% (v/v) and about 60% (v/v) alcohol, or between about 45% (v/v) and about 55% (v/v) alcohol, or about 50% (v/v) alcohol. In one embodiment, the alcohol is ethanol.

Supercritical fluid extraction using, for example, supercritical nitrogen or carbon dioxide, may also be used in accordance with the invention to obtain extracts.

Further, it will be appreciated by those skilled in the art that an extract of the invention may be subjected to one or more post extraction steps to, for example, increase or maintain the stability of the extract, modify or change the physical form of the extract or assist in formulating the extract into a composition for administration to a subject. By way of example only a liquid form extract may be lyophilised to produce a solid form of the extract.

In embodiments of the invention the extract comprises at least 15% (w/w), at least 25% (w/w), at least 35% (w/w), at least 45% (w/w), at least 50% (w/w), at least 55% (w/w), at least 65% (w/w), at least 75% (w/w) or at least 85% (w/w) bacosides. In other embodiments, the extract is a 20:1 to 30:1 extract, or about a 25:1 extract.

A commercially available extract that may be used in the present invention is that offered for sale under the trade name KeenMind® by SOHO Flordis International.

Extracts may be administered in accordance with the present invention in the form of pharmaceutical compositions, which compositions may comprise one or more pharmaceutically acceptable carriers, excipients or diluents. The compositions may be administered by any convenient or suitable route such as, for example by parenteral, oral, or topical routes. Typically, the compositions are administered via the oral route.

Pharmaceutical compositions for use in accordance with the present invention may conveniently be prepared by methods well known in the art of pharmacy. All methods include the step of bringing an extract of Bacopa Monnieri into association with one or more pharmaceutically acceptable carrier, diluent and/or excipient. In general, the compositions may be prepared by uniformly and intimately bringing into association an extract of Bacopa Monnieri with a liquid carrier or finely divided solid carrier.

Examples of pharmaceutically acceptable carriers, diluents and excipients include but are not limited to: demineralised or distilled water, saline solution, vegetable-based oils such as peanut oil, safflower oil, olive oil, cottonseed oil, maize oil and sesame oil, volatile silicones, mineral oils, cellulose derivatives such as methyl cellulose, ethyl cellulose, carboxymethylcellulose, sodium carboxymethylcellulose or hydroxypropylmethylcellulose, fatty acid esters, polyvinylpyrrolidone, carrageenan and gums. Typically the carriers, diluents and excipients will form from 5% to 99.9% by weight of the compositions. Carriers, diluents and excipients must, of course, be acceptable in the sense of being compatible with any other components of the composition and must not be deleterious to the subject.

Compositions suitable for oral administration may be presented as discrete units, such as for example gelatine or HPMC capsules, cachets or tablets, each containing a predetermined amount of extract.

When provided in the form of a capsule, the extract may be formulated with one or more pharmaceutically acceptable carriers such as starch, lactose, microcrystalline cellulose, silicon dioxide and/or a cyclic oligosaccharide such as cyclodextrin. Additional ingredients may include lubricants such as magnesium stearate and/or calcium stearate.

Tablets may be prepared by compression or moulding, optionally with one or more accessory ingredients. Compressed tablets may be prepared by compressing in a suitable machine the extract in a free-flowing form such as a powder or granules, optionally mixed with a binder, lubricant (for example magnesium stearate or calcium stearate), inert diluent or a surface active/dispersing agent. Moulded tablets may be made by moulding a mixture of the powdered extract moistened with an inert liquid diluent, in a suitable machine. The tablets may optionally be coated, for example, with an enteric coating and may be formulated so as to provide slow or controlled release of the extract therein.

Alternatively, extracts may be administered neat, i.e. in the absence of a carrier, excipient and/or diluent.

Use of an extract of Bacopa Monnieri in accordance with the invention described herein acutely improves/enhances cognitive performance in a human subject where the subject is mentally stressed, mentally fatigued and/or cognitively challenged. In embodiments of the invention, the improved or enhanced positive performance is observed and/or achieved within about 15 to 240 minutes of administration of the extract. In some embodiments, the improvement or enhancement is observed and/or achieved within in about 30, 45, 60, 90, 120, 150, 180, 210, 240, 270, 300, 330, 360, 390, or within about 420 minutes of administration of the extract.

In order to achieve the improved and/or enhanced cognitive performance, the extract may be administered to the subject prior to, during, and/or after the subject being mentally stressed, mentally fatigued and/or cognitively challenged. In some embodiments, the extract is administered at least 1 minute, at least 5 minutes, at least 10 minutes, at least 20 minutes, at least 30 minutes, at least 40 minutes, at least 50 minutes, at least 60 minutes, at least 70 minutes, at least 80 minutes, at least 90 minutes, at least 100 minutes, at least 110 minutes, at least 120 minutes, at least 130 minutes, at least 140 minutes, or at least 150 minutes, prior to the subject being mentally stressed, mentally fatigued and/or cognitively challenged. In other embodiments, the extract is administered about 5 minutes to about 3 hours prior to, or about 15 minutes to about 3 hours prior to, or about 30 minutes to about 3 hours prior to, or about 1 hour to about 3 hours prior to, or about 2 hours to about 3 hours prior to, the subject being mentally stressed, mentally fatigued and/or cognitively challenged. In further embodiments, the extract is administered up to 1 minute, up to 5 minutes, up to 10 minutes, up to 20 minutes, up to 30 minutes, up to 40 minutes, up to 50 minutes, up to 60 minutes, up to 70 minutes, up to 80 minutes, up to 90 minutes, up to 100 minutes, up to 110 minutes, up to 120 minutes, up to 130 minutes, up to 140 minutes, up to 150 minutes, up to 180 minutes, or up to 210 minutes prior to the subject being mentally stressed, mentally fatigued and/or cognitively challenged.

The subject may be mentally stressed, mentally fatigued and/or cognitively challenged as a result of undergoing a test, examination or some other activity involving cognition. Embodiments of the present invention provide improvements in cognitive performance in said tests, examinations, or other activities resulting from administration of extracts disclosed herein prior to, during, or after a subject undergoes said test, examination, or other activity. Accordingly, the methods and uses of the invention find particular application in subjects who are studying, for example high school students, university students and the like. Those skilled in the art will however recognise that the methods and uses of the invention are also applicable to subjects who are challenged in the performance of any task which involves cognition. For example, the method may find application in a subject in a work environment where the subject is required to complete a cognitively difficult and demanding task in a relatively short period of time.

In some embodiments, in order to achieve the improved and/or enhanced cognitive performance, the extract may be administered at least 5 minutes, at least 10 minutes, at least 20 minutes, at least 30 minutes, at least 40 minutes, at least 50 minutes, at least 60 minutes, at least 70 minutes, at least 80 minutes, at least 90 minutes, at least 100 minutes, at least 110 minutes, at least 120 minutes, at least 130 minutes, at least 140 minutes, or at least 150 minutes, prior to commencement of a test, examination or other activity involving cognition. In other embodiments, the extract is administered about 5 minutes to about 3 hours prior to, or about 15 minutes to about 3 hours prior to, or about 30 minutes to about 3 hours prior to, or about 1 hour to about 3 hours prior to, or about 2 hours to about 3 hours prior to commencement of a test, examination or other activity involving cognition. In further embodiments, the extract is administered up to 1 minute, up to 5 minutes, up to 10 minutes, up to 20 minutes, up to 30 minutes, up to 40 minutes, up to 50 minutes, up to 60 minutes, up to 70 minutes, up to 80 minutes, up to 90 minutes, up to 100 minutes, up to 100 minutes, up to 120 minutes, up to 130 minutes, up to 140 minutes, up to 150 minutes, up to 180 minutes, or up to 210 minutes prior to commencement of a test, examination or other activity involving cognition.

In order to achieve the improved and/or enhanced cognitive performance, the extract may be administered in an amount between about 50 mg and about 5.0 g, or in an amount between about 100 mg and about 3.0 g, or in an amount between about 100 mg and about 2.5 g, or in an amount between about 200 mg and about 2.0 g, or in an amount between about 200 mg and about 1.5 g, or in an amount between about 300 mg and about 1.5 g, or in an amount between about 400 mg and about 1.5 g, or in an amount between about 500 mg and about 1.5 g, or in an amount between about 600 mg and about 1.5 g, or in an amount between about 700 mg and about 1.5 g, or in an amount between about 800 mg and about 1.5 g, or in an amount between about 900 mg and about 1.5 g, or in an amount between about 1.0 g and about 1.5 g, or in an amount between about 1.1 g and about 1.5 g, or in an amount between about 1.2 g and about 1.5 g, or in an amount between about 1.3 g and about 1.5 g, or in an amount between about 1.4 g and about 1.5 g. The extract may be administered in an amount between about 300 mg and about 1.4 g, or in an amount between about 300 mg and about 1.3 g, or in an amount between about 300 mg and about 1.2 g, or in an amount between about 300 mg and about 1.1 g, or in an amount between about 300 mg and about 1.0 g, or in an amount between about 300 mg and about 900 mg, or in an amount between about 300 mg and about 800 mg, or in an amount between about 300 mg and about 700 mg, or in an amount between about 300 mg and about 600 mg, or in an amount between about 300 mg and about 500 mg, or in an amount between about 300 mg and about 400 mg. The extract may be administered in an amount between about 160 mg and about 960 mg, or in an amount between about 300 mg and about 750 mg, or in an amount between about 320 mg and about 640 mg. In one embodiment, the extract is administered in an amount of at least about 120 mg, in an amount of at least about 320 mg, in an amount of at least about 640 mg, or in an amount of at least about 960 mg. In one embodiment, the extract is administered in an amount of about 160 mg, about 320 mg, or about 640 mg, or about 960 mg.

The extract may be administered as a single dose or alternatively as multiple doses sequentially.

In a further aspect, the present invention provides a use of an extract of Bacopa monnieri in the manufacture of a medicament for acutely improving/enhancing cognitive performance in a human subject, wherein the subject is mentally stressed, mentally fatigued and/or cognitively challenged.

Use of an extract of Bacopa Monnieri in accordance with the invention described herein acutely improves/enhances cognitive performance in a human subject where the subject is mentally stressed, mentally fatigued and/or cognitively challenged. In embodiments of the invention, the improved or enhanced positive performance is observed and/or achieved within about 15 to 240 minutes of administration of the medicament. In some embodiments, the improvement or enhancement is observed and/or achieved within in about 30, 45, 60, 90, 120, 150, 180, 210, 240, 270, 300, 330, 360, 390, or within about 420 minutes of administration of the medicament.

In some embodiments, in order to achieve the improved and/or enhanced cognitive performance, the medicament may be administered at least 5 minutes, at least 10 minutes, at least 20 minutes, at least 30 minutes, at least 40 minutes, at least 50 minutes, at least 60 minutes, at least 70 minutes, at least 80 minutes, at least 90 minutes, at least 100 minutes, at least 110 minutes, at least 120 minutes, at least 130 minutes, at least 140 minutes, or at least 150 minutes, prior to commencement of a test, examination or other activity involving cognition. In other embodiments, the medicament is administered about 5 minutes to about 3 hours prior to, or about 15 minutes to about 3 hours prior to, or about 30 minutes to about 3 hours prior to, or about 1 hour to about 3 hours prior to, or about 2 hours to about 3 hours prior to commencement of a test, examination or other activity involving cognition. In further embodiments, the medicament is administered up to 1 minute, up to 5 minutes, up to 10 minutes, up to 20 minutes, up to 30 minutes, up to 40 minutes, up to 50 minutes, up to 60 minutes, up to 70 minutes, up to 80 minutes, up to 90 minutes, up to 100 minutes, up to 110 minutes, up to 120 minutes, up to 130 minutes, up to 140 minutes, up to 150 minutes, up to 180 minutes, or up to 210 minutes prior to commencement of a test, examination or other activity involving cognition.

In an embodiment, in order to achieve the improved and/or enhanced cognitive performance, the medicament comprises an amount of extract of between about 50 mg and about 5.0 g, or in an of extract of amount between about 100 mg and about 3.0 g, or in an of extract of amount between about 100 mg and about 2.5 g, or in an amount of extract of between about 200 mg and about 2.0 g, or in an amount of extract of between about 200 mg and about 1.5 g, or in an amount of extract of between about 300 mg and about 1.5 g, or in an amount between of extract of about 400 mg and about 1.5 g, or in an amount of extract of between about 500 mg and about 1.5 g, or in an amount of extract of between about 600 mg and about 1.5 g, or in an amount of extract of between about 700 mg and about 1.5 g, or in an amount of extract of between about 800 mg and about 1.5 g, or in an amount of extract of between about 900 mg and about 1.5 g, or in an amount of extract of between about 1.0 g and about 1.5 g, or in an amount of extract of between about 1.1 g and about 1.5 g, or in an amount of extract of between about 1.2 g and about 1.5 g, or in an amount of extract of between about 1.3 g and about 1.5 g, or in an amount of extract of between about 1.4 g and about 1.5 g. The medicament may be administered in an amount of extract of between about 300 mg and about 1.4 g, or in an amount of extract of about 300 mg and about 1.3 g, or in an amount of extract of between about 300 mg and about 1.2 g, or in an amount of extract of between about 300 mg and about 1.1 g, or in an amount of extract of between about 300 mg and about 1.0 g, or in an amount of extract of between about 300 mg and about 900 mg, or in an amount of extract of between about 300 mg and about 800 mg, or in an amount of extract of between about 300 mg and about 700 mg, or in an amount of extract of between about 300 mg and about 600 mg, or in an amount of extract of between about 300 mg and about 500 mg, or in an amount of extract of between about 300 mg and about 400 mg. The medicament may be administered in an amount of extract of between about 160 mg and about 960 mg, or in an amount of extract of between about 300 mg and about 750 mg, or in an amount of extract of between about 320 mg and about 640 mg. In one embodiment, the medicament is administered in an amount of extract of at least about 120 mg, in an amount of extract of at least about 320 mg, in an amount of extract of at least about 640 mg, or in an amount of extract of at least about 960 mg. In one embodiment, the medicament is administered in an amount of extract of about 160 mg, about 320 mg, or about 640 mg, or about 960 mg.

The medicament may be administered as a single dose or alternatively as multiple doses sequentially.

This invention may also be said broadly to consist in the parts, elements and features referred to or indicated in the specification of the application, individually or collectively, and any or all combinations of any two or more said parts, elements or features, and where specific integers are mentioned herein which have known equivalents in the art to which this invention relates, such known equivalents are deemed to be incorporated herein as if individually set forth.

### EXAMPLES

The invention will now be described in more detail, by way of illustration only, with respect to the following examples. The examples are intended to serve to illustrate this invention and should in no way be construed as limiting the generality of the disclosure of the description throughout this specification.

The following clinical study was carried out which demonstrates the invention.

### Example 1

### Method

### Participants

Twenty-four healthy volunteers (4 males and 20 females) aged between 18 and 56 years (mean±standard deviation=25.25±9.30), with Body Mass Indicies ranging from 15.40 to 32.74 kg/m² (23.48±4.39) were recruited for the study. Participants were restricted from taking part based on several self-report screening criteria which included the following: individuals who smoke, had any history of psychiatric disorders or neurological diseases, individuals suffering from endocrine, gastrointestinal or bleeding disorders, individuals with chronic illness and infection, individuals who were pregnant or lactating were restricted from taking part. Any individuals taking any medications or herbal extracts were also excluded from participation. On the day of testing participants were required to consume only a light breakfast while abstaining from alcohol and coffee. The study was approved by the Swinburne University Human Research Ethics Committee and was registered with the Australian and New Zealand Clinical Trials Registry (ACTRN12612000810819).

### Treatment and Study Design

A double-blind, placebo-controlled, crossover design was employed for this study. On each testing day participants received four capsules containing an inert placebo, 320 mg of KeenMind® (CDRI 08) Bacopa monnieri (BM) extract or 640 mg of KeenMind® (CDRI 08) BM extract. KeenMind® (CDRI 08) is standardized for no less than 55% of total bacosides. Each capsule contained 160 mg BM extract (25:1) equivalent to 4 g of dried herb. The extract of KeenMind® (CDRI 08) BM was prepared from stems, leaves and roots of a cultured variety of BM collected from West Bengal and extracted with 50% ethanol. The placebo capsule was identical in shape, smell, taste and weight and was supplied in the form of four 160 mg capsules (made up of inert plant based materials) per participant per testing day. Randomization was performed using a computer generated randomization program that enables equal probability of being allocated to one of the three treatment conditions at each visit.

### Cognitive Demand Battery (CDB)

The CDB comprised of a “stress and mental fatigue” visual analogue scale, two Serial subtraction tasks (Serial Threes and Serial Sevens) and the Bakan Rapid Visual Information Processing task which were all administered on computers running MS Windows®. The individual tasks are described below.

### (1) “Stress and Mental Fatigue” Visual Analogue Scales (VAS):

Participants indicated their current subjective feeling of stress and mental fatigue by clicking on the 100 mm visual analogue scale line. The left hand end point of this line was labelled “not at all” and the right hand end point was labelled “very much so”. One minute was allowed to complete the VAS before and after the CDB.

### (2) Serial 3's Subtraction Task:

Participants were required to mentally count backwards in threes from a given number as accurately and as quickly as possible for duration of two minutes. A random starting number between 800 and 999 was presented on the computer screen, which was cleared by the entry of the first response. The three-digit number responses were recorded via the numeric keypad provided. This was displayed on the monitor of a desktop computer. Responses entered by the participants appeared on screen, masked by three asterisks. Pressing the “enter” key signalled the end of each response and cleared the three asterisks in the box ready for the next three-digit number. The task was scored on number of correct responses.

### (3) Serial 7's Subtractions Task:

Participants were required to complete a task which was identical to the Serial Threes task in its set up and duration. However, the only noticeable distinction was the subtraction of sevens replacing the subtraction of threes.

### (4) Rapid Visual Information Processing Task (RVIP):

Participants were required to monitor a randomised continuous series of digits for targets of three consecutive odd or even numbers. The digits were presented at a rate of 100 per minute with eight correct target strings presented in each minute. Participants responded to the detection of target string by pressing the “space bar” as quickly as possible. The task was scored by calculating the correct number of target strings identified and the reaction time for correct detection. The task lasted for five minutes. The total duration of each cycle of the CDB (Serial Threes, Serial Sevens, and RVIP) was 10 minutes.

### Blood Pressure

Brachial blood pressure was calculated in the morning with the participant seated and following a 5 minute rest period. All measurements were calculated using an automatic sphygmomanometer designed for professional use (Omron, 705IT) and validated according to both the European Hypertension Society (EHS) and the British Hypertension Society (BHS) protocols. Measurements were completed using an appropriately sized cuff by an experienced research assistant and a cardiac technologist. The mean arterial pressure (MAP) was calculated according to the following formula (2*DBPpSBP)/3. Pulse pressure and augmentation index PP and augmentation index were calculated centrally using a non-invasive device (SphygmoCor; AtCor Medical, Sydney, Australia) by means of applanation tonometry. Through a mathematical transfer function, SphygmoCor derived the ascending aortic waveform from a recording of the radial artery before automatically calculating a range of cardiovascular parameters indicative of arterial stiffness. PP was automatically calculated by deducting the central diastolic pressure from the central systolic pressure, whereas central augmentation index was calculated by dividing the augmentation pressure by the PP, multiplied by 100. All recordings were completed with the participant sitting down whilst their arm rested on a table with their palm facing upwards. To ensure the reliability of analysis, only recordings with an operator index equal to or greater than 85 were utilized in statistical analysis. The cardiovascular parameters derived by SphygmoCor have been previously observed to be related to cognitive performance.

### Procedure

Each participant was required to attend a total of four sessions (one practice visit and three study visits) that were conducted one week apart to ensure sufficient wash out between each acute condition. Participants were asked to consume a light breakfast (e.g., one standard serve of cereal or two pieces of toast at home on each testing day) before arriving at the testing location. Testing took place in a suite of dedicated university laboratories at the Swinburne Centre for Human Psychopharmacology. Prior to the first study visit, participants completed three cycles of the CDB. This was to control for practice effects as well as to allow for familiarization with the test battery and procedures that would be carried out during study visits. The practice day data were not included in any analyses.

Upon arriving at the laboratory, participants completed one cycle of the CDB. This was followed by measurements for participants' blood pressure, arterial stiffness and cerebral blood flow. The ten minute CDB as well as cardiovascular measurements (blood pressure, arterial stiffness, and cerebral blood flow) was completed pre-dose to establish baseline performance. This was followed immediately by ingestion of the allocated treatment tablets. Participants then waited two hours following the consumption of the tablet (during which participants were instructed to consume nothing other than water and to avoid any strenuous exercise). After the two hour waiting time, participants completed a continuous series of six CDB cycles which took approximately 60 minutes. Then participants' blood pressure, arterial stiffness and cerebral blood flow were measured. The same testing sequence was carried out in all three study visits.

### Data Treatment and Statistics

All scores were within acceptable range for these variables and were therefore subjected to parametric analyses. Repeated measures ANOVAs were conducted on each of the main variables with seven time conditions (baseline and cycles 1 to 6) and Treatment (Placebo, 320 mg and 640 mg) conditions. Therefore several 3×6 repeated measures ANOVAs were employed to compare change from baseline differences between the three treatment conditions for the CDB variables. Paired-sample t-tests were utilised when a significant interaction occurred between time and treatment to explore differences between performance according to treatment. Additional repeated measures ANOVAs were conducted on both the VAS scale and cardiovascular measures with two time conditions (baseline and post treatment) and three treatment conditions (Placebo, 320 mg and 640 mg). Therefore several 2×3 repeated measures ANOVAs were employed to compare differences between these variables. All statistical tests were two-tailed and alpha was set at 0.05.

### Results

No adverse effects were reported throughout the study for any of the three treatments. Prior to examination of the cognitive tests, all data was examined with regard to gender and treatment order effects, with no significant pattern of results emerging. Baseline performance scores (mean and standard error) for the CDB and difference from baseline scores for each outcome measure and treatment are summarized in Table 1 below. Significant time, treatment and time×treatment effects are reported in the text below; non-significant effects are not reported for the sake of brevity.

TABLE 1
Mean (±SE) baseline scores and change from baseline scores for six repetitions of the CDB.

| | | Baseline | 1^{st} repetition | 2^{nd} repetition | 3^{rd} repetition | 4^{th} repetition | 5^{th} repetition | 6^{th} repetition |
|---|---|---|---|---|---|---|---|---|
| Serial 3s: | Placebo | 37.04 ± 3.24 | 0.04 | 0.92 | 3.46 | 1.25 | 7.63 | 9.54 |
| No correct | 320 mg | 36.30 ± 4.05 | 4.27 | 5.50 | 5.68 | 9.09 | 8.73 | 7.95 |
| | 640 mg | 37.92 ± 3.30 | 1.50 | 2.71 | 4.21 | 5.29 | 5.38 | 5.79 |
| Serial 3s: | Placebo | 2.12 ± 0.38 | 0.46 | 0.25 | 0.67 | 2.54 | 0.25 | 0.21 |
| No incorrect | 320 mg | 1.87 ± 0.48 | −0.59 | 0.41 | −0.14 | 0.45 | 0.45 | 0.09 |
| | 640 mg | 1.92 ± 0.40 | 0.17 | −0.46 | 0.04 | 0.88 | 0.67 | 1.21 |
| Serial 7s: | Placebo | 19.33 ± 2.06 | 2.29 | 2.17 | 3.83 | 3.38 | 6.63 | 6.38 |
| No correct | 320 mg | 20.04 ± 2.12 | 1.55 | 2.18 | 5.05 | 4.95 | 6.86 | 5.95 |
| | 640 mg | 18.92 ± 1.77 | 2.63 | 3.75 | 3.04 | 5.79 | 4.79 | 5.33 |
| Serial 7s: | Placebo | 2.29 ± 0.35 | −0.54 | 0.08 | 0.25 | 0.54 | −0.08 | 0.25 |
| No incorrect | 320 mg | 1.83 ± 0.31 | 0.73 | 0.95 | −0.09 | 0.18 | −0.05 | 0.14 |
| | 640 mg | 2.46 ± 0.48 | −0.96 | −0.29 | 0.13 | −0.29 | 0.25 | 0.38 |
| RVIP | Placebo | 17.13 ± 1.96 | −1.00 | 0.04 | 0.96 | −1.83 | −1.46 | −0.96 |
| correct | 320 mg | 16.26 ± 1.91 | 0.05 | −0.23 | 0.64 | −0.36 | −1.05 | −0.55 |
| | 640 mg | 15.88 ± 2.27 | −0.83 | −0.88 | 0.13 | −1.96 | −1.83 | −1.25 |
| RVIP | Placebo | 11.67 ± 3.78 | 0.25 | −2.42 | −0.88 | −2.88 | −2.21 | −1.75 |
| False alarms | 320 mg | 13.26 ± 4.68 | −1.50 | −0.86 | −1.27 | −2.41 | −2.95 | −4.68 |
| | 640 mg | 11.42 ± 4.03 | 0.92 | 2.75 | −2.25 | −0.79 | −1.88 | −0.50 |
| RVIP | Placebo | 496.81 ± 12.43 | −10.36 | 8.66 | −8.34 | 0.12 | −2.88 | −8.09 |
| reaction time | 320 mg | 467.62 ± 14.75 | 16.51 | 28.54 | 36.78 | 38.96 | 22.35 | 44.92 |
| | 640 mg | 464.31 ± 24.56 | 1.91 | 24.62 | 33.32 | 20.15 | 20.52 | 20.56 |

### Cognitive Demand Battery

Serial 3s.

A significant time×treatment interaction (F_{10, 210}=1.89, p<0.05) emerged for the number of correct responses within the Serial 3s task. Examination of the change from baseline scores at each assessment repetition revealed significantly better performance within the 320 mg BM treatment in comparison to placebo during the 1^{st} repetition, t (21)=2.05, p=0.05, 4^{th} repetition, t (21)=2.48, p=0.02, and strongly trending towards the same effect during the 2^{nd} repetition, t (21)=2.02, p=0.056. A significant main effect for time (F_{5, 105}=6.06, p<0.001) was also observed, with more correct responses occurring across the repeated assessments. No modulation of the number of incorrect responses was evident across the assessment period.

Serial 7s.

A trend towards a time×treatment interaction was observed (F_{10, 210}=1.76, p=0.07) for the number of incorrect responses for Serial 7s performance. Examination of the change from baseline scores indicated that this trend was largely driven by improved performance in the 640 mg BM treatment condition in comparison to the 320 mg BM condition, t (21)=2.10, p<0.05, during the 1st repetition. A main effect of time was also observed, (F_{5, 105}=4.36, p=0.001) for the number of correct responses, with the number of correct responses increasing across the repeated assessments.

RVIP.

No significant alterations in performance by treatment or over time were observed for the RVIP task.

### Visual Analogues Scales—Stress and Fatigue Ratings

Assessment of participants' ratings of stress and fatigue were taken prior to, and after completion of the baseline performance of the CDB. Completion of the battery induced an increase in ratings of fatigue across the three conditions evidenced by a significant effect of time, F_{1, 22}=7.32, p=0.013. Following the 2-hour break after treatment consumption, participants ratings of fatigue and stress were again taken prior to, and after completion of the CDB (6 repetitions). Completion of this extended battery induced a significant increase in ratings of stress (F_{1, 22}=8.74, p<0.01) and fatigue (F_{1, 22}=67.30, p<0.001). The interaction between treatment and time for both stress (F_{2, 44}=0.31, p>0.05) and fatigue (F_{2, 44}=1.74, p>0.05) were not significant, indicating that neither treatments attenuated the stress or fatigue inducing effects of the CDB. Mean scores (±SE) for the four completions of the visual analogue scales appear in Table 2 below.

TABLE 2
Mean (±SE) scores for the stress and fatigue measures for the three
treatment conditions pre- and post-dosing and pre- and post-CDB completion

| | Placebo | 320 mg Bacopa | 640 mg Bacopa | Placebo | 320 mg Bacopa | 640 mg Bacopa |
|---|---|---|---|---|---|---|
| | Pre-dose | | | Post-dose | | |
| VAS: Stress rating | 31.08 ± 4.25 | 23.87 ± 3.64 | 23.38 ± 4.03 | 28.08 ± 4.14 | 25.00 ± 3.87 | 28.92 ± 4.48 |
| pre-CDB | | | | | | |
| VAS: Stress rating | 31.04 ± 4.03 | 26.87 ± 3.80 | 28.63 ± 4.21 | 39.17 ± 5.35 | 34.00 ± 5.51 | 35.00 ± 4.77 |
| post-CDB | | | | | | |
| VAS: Fatigue rating | 31.79 ± 3.69 | 34.43 ± 4.11 | 38.33 ± 4.37 | 35.17 ± 3.80 | 40.96 ± 5.16 | 43.54 ± 5.04 |
| pre-CDB | | | | | | |
| VAS: Fatigue rating | 39.92 ± 4.17 | 41.43 ± 5.22 | 43.50 ± 5.17 | 60.00 ± 5.74 | 58.52 ± 6.41 | 69.42 ± 4.22 |
| post-CDB | | | | | | |

### Cardiovascular Measurements

No significant change in blood pressure (central and brachial assessments) or in the Augmentation index was observed between the three treatments. Mean values (±SE) for the cardiovascular assessments appear in Table 3 below.

TABLE 3
Mean (±SE) scores for the cardiovascular measures for the three treatment
conditions pre- and post-dosing and pre- and post-CDB completion

| | Placebo | 320 mg Bacopa | 640 mg Bacopa | Placebo | 320 mg Bacopa | 640 mg Bacopa |
|---|---|---|---|---|---|---|
| | Pre-dose | | | Post-dose | | |
| Augmentation index | 11.64 ± 2.09 | 10.47 ± 9.74 | 9.74 ± 2.81 | 10.95 ± 2.31 | 12.86 ± 2.33 | 14.46 ± 2.24 |
| Diastolic BP | 73.29 ± 1.37 | 71.33 ± 1.61 | 71.81 ± 2.03 | 72.05 ± 2.13 | 73.62 ± 1.62 | 75.57 ± 1.87 |
| Systolic BP | 102.95 ± 2.31 | 101.91 ± 2.15 | 101.95 ± 2.78 | 101.19 ± 2.38 | 102.00 ± 2.12 | 104.24 ± 2.80 |
| Brachial Diastolic BP | 72.14 ± 1.36 | 70.19 ± 1.58 | 70.76 ± 1.97 | 70.76 ± 1.97 | 72.57 ± 1.58 | 74.57 ± 1.86 |
| Brachial Systolic BP | 117.57 ± 2.89 | 116.38 ± 2.19 | 116.71 ± 3.12 | 115.91 ± 2.60 | 115.90 ± 2.27 | 117.00 ± 2.89 |

### Discussion

Assessment of the change from baseline performance following consumption of placebo, 320 mg BM, 640 mg BM revealed performance on the Serial 3s subtraction test was improved at the first, second, and fourth repetition post-dosing in the 320 mg BM condition. Additionally, during the first repetition of the Serial 7s subtraction task, a significantly lesser number of incorrect responses were provided in the 640 mg BM condition in comparison to the 320 mg BM condition. Completion of the CDB generally increased participants' ratings of stress and fatigue, and these effects were not attenuated by consumption of either dose of BM. No significant differences between conditions were identified upon the measures of cardiovascular functioning, possibly indicating that at the doses assessed and the timeline of the cardiovascular assessments that BM had no obvious acute effect upon cardiovascular parameters.

Consumption of the clinically standard dose of BM (320 mg; CDRI 08) improved Serial 3s performance in three of the first four repetitions of the CDB. This improvement from baseline in the correct number of subtractions ranged from two to eight more correct subtractions within the 320 mg BM condition in comparison to placebo across these four repetitions. The largest discrepancy in performance between the conditions occurred at the 4th repetition, possibly indicating an optimal effect of the clinically standard dose at this time-point when participants would be expected to be reaching highest levels of stress and fatigue.

### Example 2

### Method

### Participants

Twenty healthy volunteers were selected as set out in example 1. The Body Mass Indicies of the participants ranged from 14.8 to 32 kg/m² (22.84±3.09).

### Treatment and Study Design

The study was an acute, 4-arm, randomised, double-blind, placebo-controlled crossover design. Participants attended 4 testing sessions where they completed cognitive, mood and stress assessments, prior to and 1, 2 and 4 hours post supplementation. Participants orally consumed each treatment on each occasion, directly after a light meal. Each treatment was administered after a 1 week wash out period. On each testing day participants received six capsules containing an inert placebo, 320 mg of KeenMind® (CDRI 08) Bacopa monnieri (BM) extract, or 640 mg of KeenMind® (CDRI 08) BM extract, or 960 mg of KeenMind® (CDRI 08) BM extract. KeenMind® (CDRI 08) is standardised for no less than 55% of total bacosides. Each capsule contained 160 mg BM extract (25:1) equivalent to 4 g of dried herb. The extract of KeenMind® (CDRI 08) BM was prepared from stems, leaves and roots of a cultured variety of BM collected from West Bengal and extracted with 50% ethanol. The placebo capsule was identical in shape, smell, taste and weight and was supplied in the form of four 160 mg capsules (made up of inert plant based materials) per participant per testing day. Randomisation was performed using a computer generated randomisation program that enables equal probability of being allocated to one of the four conditions at each visit.

### Cognitive Performance

The Purple Multitasking Framework (MTF) comprises four tasks that were administered at the same time to increase feelings of stress and to divide attentional resources so that each participant was working to their absolute maximal acute ability.

The Purple MTF (previously Defined Intensity Stressor Simulator—DISS) battery has been developed as a platform for eliciting acute psychological stress. Previous research has shown that performance of the DISS battery reliably engenders increases in self-ratings of negative mood and anxiety, and engenders stress-related physiological responses. The specific advantages of this system over other laboratory stressors (such as simulated public speaking) were that it can be repeated on a number of occasions, allowing its use in crossover design experiments, and that it produces a number of outcomes which allow a concomitant assessment of psychomotor, memory and attentional performance.

The platform has been used successfully in several studies examining the stress-relieving effects of natural products including herbal extracts and chewing gum. There are further unpublished data demonstrating similar effects.

Stress can be described in terms of subjective experience and physiological responses, the latter involve activation of the hypothalamic-pituitary-adrenocortical (HPA) axis and/or the sympathetic arm of the autonomic nervous system. All three dimensions of stress can be measured in the laboratory.

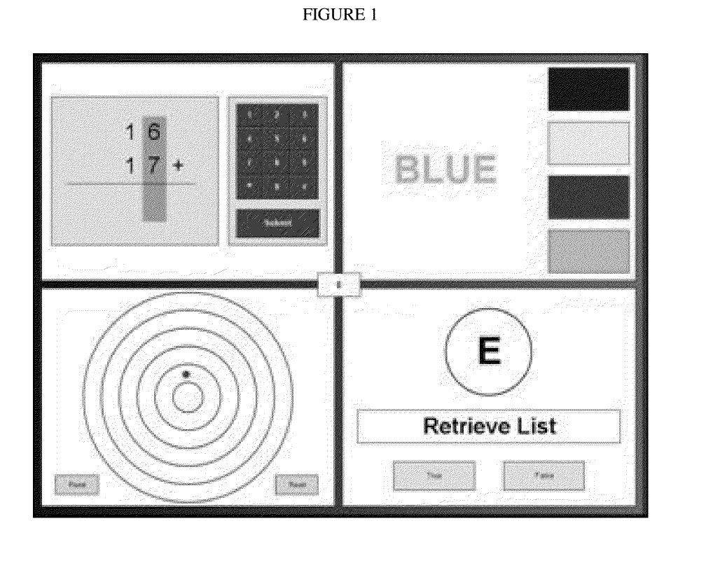

The tasks used in the cognitive performance assessment were the mental arithmetic task; the stroop task; the tracking task; and the memory search task. These tasks are illustrated in FIG. 1. All responses were made with an external mouse. In this instance, a 20 min version of the platform was employed, with participants constantly monitored by research staff to increase performance anxiety (social evaluation reliably increases stress).

Participants were instructed via on screen standard instructions to attend simultaneously to all four tasks, while monitoring the central counter displaying their accumulated aggregate score. Accuracy and speed of response dictate the score, with failure to respond resulting in negative scoring. Throughout completion of the battery a researcher was positioned within the peripheral vision of the participant, seemingly monitoring performance throughout.

Previous research has shown that performance of the platform is sensitive to a number of interventions, and that it engenders increases in self-ratings of negative mood, anxiety and stress-related physiological responses. In order to measure the mood effects and physiological stress-response of completion of the DISS, and subsequent modulation of these effects by the treatments, mood was assessed with Bond-Lader scales and the STAI ‘state’ subscale before and after each completion of the battery.

The Purple MTF is unique amongst laboratory stressors in that it is suitable as a repeated measure. It also provided performance measures for the individual tasks and an overall performance score. In general, successful performance on the DISS battery requires concentration and can be viewed as being a measure of executive functioning and working memory performance.

### Mental Arithmetic

This task requires participants to perform a series of arithmetic (additions) problems. Using a number pad on the right, participants used the mouse to click on the number in which they thought should go in the right column, and work through the sum, completing all columns, and pressing done. Participants were awarded 10 points for a correct answer and 10 points were subtracted for an incorrect answer.

### Stroop

The Stroop task is a classic psychological test of selective attention and response inhibition. In this task, a series of words were presented (Red, Blue, Yellow and Green) in differing colours (Red, Blue, Yellow and Green). Participants were asked to click one of four coloured blocks on the right hand side of the task in response to the colour of the font, regardless of the meaning of the word. For example, if the colour name ‘blue’ appeared in red font, the correct response was to click on the ‘Red’ colour block on the right. 10 points were awarded for every colour word that was correctly identified, and 10 points were subtracted for each incorrect answer, or for not making a response in the allotted time period (a ‘timeout’).

### Memory Search

An array of letters was presented to the participants to remember. The letters disappear after 4 seconds but could be viewed again by clicking on “retrieve list” button. Approximately every 10 seconds, a single target letter appeared. Participants were instructed to indicate whether the target letter had appeared in the original list of four letters by clicking on the “yes” or “no” buttons. Ten points were awarded for a correct answer, 10 points deducted for an incorrect answer or no response, and 5 points were deducted every time the list was retrieved.

### Visual Tracking

This task assesses psychomotor ability. A small dot drifted outwards from the centre of a target comprising five concentric circles. The participants were instructed to allow the dot to travel as far out of the centre as possible, without letting it hit the edge of the target, before clicking on the “reset” button. Two points were added to the running total for every circle that the dot passed through (with a maximum of 10 points), with a penalty of 10 points for every half second that passes between the dot hitting the outer edge and the participant clicking on the “reset” button.

### Mood

The mood effects are important in better understanding the subjective effects of acute doses of KeenMind®. These ratings can be more reliable than the cognitive scores, particularly when assessing results obtained with lower participant numbers. Participants were assessed on the criteria of stress, fatigue, alertness, contentedness (feeling of well-being), and calmness.

The State-Trait Anxiety Inventory (STAI) comprises of two scales. The ‘State’ (STAI-S) subscale is a widely used instrument for measuring fluctuating levels of anxiety. The subscale contains 20 statements (e.g. ‘I am calm’). Participants rated how much they feel like each statement at the time of making the response by marking a 4-point scale ranging from ‘not at all’ to ‘very much so’. The ‘Trait’ (STAI-T) subscale comprises 20 different statements (e.g. ‘Some unimportant thought runs through my mind and bothers me’). Participants were asked to indicate how they generally feel on a scale ranging from ‘almost never’ to ‘almost always’. Scores on both sections of the STAI range from 20 to 80, with higher scores indicating more anxiety. The Trait subscale of the STAI can be used as a screening measure at baseline in order to detect those participants who may have excessive levels of trait anxiety prior to commencing the study. The State subscale of the STAI was subsequently used at each study visit both before and after the Purple Multi-tasking Framework in order to measure acute levels of anxiety in response to a stressor. Higher scores indicate greater anxiety. For each completion of the stress battery the pre-Purple Multi-Tasking Framework factor scores were subtracted from the post-Purple Multi-Tasking Framework factor scores to give single scores representing the change in mood engendered by completion of the Purple Multi-Tasking Framework battery.

### Procedure

Each participant was required to attend a total of five sessions (one practice visit and four study visits) that were conducted one week apart to ensure sufficient wash out between each acute condition. Participants were asked to consume a light breakfast (e.g., one standard serve of cereal or two pieces of toast at home on each testing day) before arriving at the testing location. Testing took place in a suite of dedicated university laboratories at the Swinburne Centre for Human Psychopharmacology. Prior to the first study visit, participants completed three cycles of the Purple Multi-Tasking Framework. This was to control for practice effects as well as to allow for familiarisation with the test battery and procedures that would be carried out during study visits. The practice day data were not included in any analyses.

The participants received four treatments: treatment 1—Placebo; treatment 2—320 mg of KeenMind®, treatment 3—640 mg of KeenMind®, and treatment 4—960 mg of KeenMind®.

TABLE 4
Flow chart of treatment and testing sessions.

| | | Weekly |
|---|---|---|
| | Screening | testing |
| | visit | sessions |
| Study Plan | (V1) | (V2-V5) |
| History/Participant screening/Exclusion | ✓ | |
| criteria | | |
| Demographics Questionnaire | ✓ | |
| PURPLE Multitasking Framework (PMF) | ✓ | ✓ |
| Bond-Lader Visual Analogue Scales | | ✓ |
| State Trait Anxiety Index | ✓ | ✓ |
| Stress and Fatigue Visual Analogue Mood | | ✓ |
| Scales (VAMS) | | |
| Symptom Checklist | | ✓ |

### Data Treatment and Statistics

All variables were within acceptable range for these variables and were therefore subjected to parametric analyses. Repeated measures ANOVAs were conducted on each of the main variables. For the cognitive variables derived from the Purple MTF the repeated measures ANOVAs were 4 levels of time (baseline, 1 hour, 2 hours and 4 hours post-baseline) by 4 levels of treatment (placebo 320, 640 and 960 mg of Keenmind®). A different 4×4 ANOVA was computed for each of the cognitive variables. For mood this comprised 4 levels of time (baseline, 1 hour, 2 hours and 4 hours post-baseline), 2 levels of task (before and after Purple MTF) and 4 levels of treatment (placebo 320, 640 and 960 mg of Keenmind®). Paired-sample t-tests were utilised where appropriate to explore differences between performance according to treatment.

### Results

No adverse effects were reported throughout the study for any of the three treatments. Prior to examination of the cognitive tests, all data was examined with regard to gender and treatment order effects, with no significant pattern of results emerging.

1. Cognitive Performance

(a) Mental Arithmetic Task

The clearest effect compared to placebo was the change from baseline to 1 and 4 hours for the 320 mg dose. There was a significant improvement in reaction time for this task over these durations.

(b) Stroop Task

The effects compared to placebo were the change from baseline to 1, 2 and 4 hours for the 320 mg dose. This suggests improved attention for the 320 mg condition compared to the placebo.

(c) Tracking Task

The clearest effect compared to the placebo was the change from baseline to 4 hours for the 640 mg condition. This suggests an improvement in visual processing and hand-eye coordination.

(d) Memory Search Task

The clearest effects compared to placebo was the change from baseline to 2 hours for the 960 mg dose and for the change from baseline to the 2 hours for the 320 mg dose. This suggests improved information processing and working memory due to the 960 mg and 320 mg doses.

2. Mood

(a) Stress

The largest effect was a due to administration of a dose of 320 mg after 4 hours. Overall there was an increase in stress over time due to the task, which is expected. However the 320 mg dose mitigated this overall increase in stress.

(b) Fatigue

There was a large increase in feelings of fatigue throughout the study conditions. This was again expected due to the heavy load of testing imposed on each participant and repeat testing over each day of testing. All doses (320 mg, 640 mg, and 960 mg) of KeenMind® mitigated this effect compared to placebo

(c) Alertness

There was also a decrease in alertness over each testing session. Again, this is to be expected due to the testing regime utilised. The 960 mg dose mitigated this decrease in alertness and was most pronounced effect at 1 hour suggesting that the effect may not be sustained pastl hour even at the highest dose.

(d) Contentedness

Contentedness usually measures feelings of well-being. Subjective reporting of contentedness decreased during each day. The only effect observed was for the 960 mg dose compared to the placebo, in which an improvement was observed at 1 hour post-baseline.

(e) Calmness

There were no improvements in this variable due to any of the treatments.

### CONCLUSION

Overall the data show similar and stronger effects than those shown in Example 1 with a good correspondence between the cognitive and subjective mood data. Results generally replicated and extended the results shown in Example 1. Cognitive Performance: compared to the placebo condition: the 320 mg dose improved mental arithmetic, stroop and memory search tasks; a 640 mg dose improved the tracking task; a 960 mg dose improved performance on the memory search task. In terms of the mood effects: compared to the placebo: the 320 mg dose decreased the experience of stress; the 640 mg dose improved feelings of fatigue; the 960 mg dose improved feelings of fatigue, increased alertness and content.

Generally, all doses administered to the participants improved and/or enhanced cognitive performance while they were stressed, mentally fatigued and/or cognitively challenged.

Those skilled in the art will appreciate that the invention described herein is susceptible to variations and modifications other than those specifically described. It is to be understood that the invention includes all such variations and modifications.

The reference in this specification to any prior publication (or information derived from the prior publication), or to any matter which is known, is not, and should not be taken as an acknowledgment or admission or any form of suggestion that the prior publication (or information derived from the prior publication) or known matter forms part of the common general knowledge in the field of endeavour to which this specification relates.

The claims defining the invention are as follows:

1. A method for acutely improving/enhancing cognitive performance in a human subject, wherein the subject is mentally stressed, mentally fatigued and/or cognitively challenged, the method comprising administration to the subject of an extract of Bacopa monnieri.

2. The method of claim 1, wherein the extract is prepared from stems, leaves and roots of Bacopa monnieri.

3. The method of claim 1 or claim 2, wherein the extract is an alcoholic extract.

4. The method of claim 3, wherein the alcoholic extract is an aqueous alcoholic extract.

5. The method of claim 4, wherein the aqueous alcoholic extract is an aqueous C₁-C₆ alcoholic extract.

6. The method of claim 5, wherein the aqueous C₁-C₆ alcoholic extract is a 50% (v/v) aqueous C₁-C₆ alcoholic extract.

7. The method of claim 6, wherein the 50% (v/v) aqueous C₁-C₆ alcoholic extract is a 50% (v/v) aqueous ethanolic extract.

8. The method of any one of claims 1 to 7, wherein the extract comprises at least 55% (w/w) bacosides.

9. The method of any one of claims 1 to 8, wherein the extract is administered to the subject prior to, during, or after the subject being mentally stressed, mentally fatigued and/or cognitively challenged.

10. The method of claim 9, wherein the extract is administered to the subject prior to the subject being mentally stressed, mentally fatigued and/or cognitively challenged.

11. The method of claim 10, wherein the extract is administered at least or about 15 minutes prior to, at least or about 30 minutes prior to, at least or about 1 hour prior to, or at least or about 2 hours prior to, the subject being mentally stressed, mentally fatigued and/or cognitively challenged.

12. The method of any one of claims 1 to 8, wherein the subject is mentally stressed, mentally fatigued and/or cognitively challenged as a result of undergoing a test, examination or some other activity involving cognition.

13. The method of claim 12, wherein the extract is administered prior to commencement of the test, examination or other activity involving cognition.

14. The method of claim 13, wherein the extract is administered at least or about 15 minutes prior to, at least or about 30 minutes prior to, at least or about 1 hour prior to, or at least or about 2 hours prior to, commencement of the test, examination or other activity involving cognition.

15. The method of any one of claims 1 to 14, wherein the extract is administered in an amount between about 200 mg and about 2.0 g, or in an amount between about 300 mg and about 1.0 g, or in an amount between about 320 mg and about 960 mg, or in an amount between about 320 mg and about 640 mg.

16. The method of any one of claims 1 to 14, wherein the extract is administered in an amount of at least about 320 mg, or in an amount of at least about 640 mg, or in an amount of at least about 960 mg.

17. The method of any one of claims 1 to 16, wherein the extract is administered orally.

18. Use of an extract of Bacopa monnieri for acutely improving/enhancing cognitive performance in a human subject, wherein the subject is mentally stressed, mentally fatigued and/or cognitively challenged.

19. Use of an extract of Bacopa monnieri in the manufacture of a medicament for acutely improving/enhancing cognitive performance in a human subject, wherein the subject is mentally stressed, mentally fatigued and/or cognitively challenged.

20. The use of claim 18 or claim 19, wherein the extract is prepared from stems, leaves and roots of Bacopa monnieri.

21. The use of any one of claims 18 to 20, wherein the extract is an alcoholic extract.

22. The use of claim 21, wherein the alcoholic extract is an aqueous alcoholic extract.

23. The use of claim 22, wherein the aqueous alcoholic extract is an aqueous C₁-C₆ alcoholic extract.

24. The use of claim 23, wherein the aqueous C₁-C₆ alcoholic extract is a 50% (v/v) aqueous C₁-C₆ alcoholic extract.

25. The use of claim 24, wherein the 50% (v/v) aqueous C₁-C₆ alcoholic extract is a 50% (v/v) aqueous ethanolic extract.

26. The use of any one of claims 18 to 25, wherein the extract comprises at least 55% (w/w) bacosides.

27. The use of any one of claims 18 to 26, wherein the extract or medicament is administered to the subject prior to, during, or after the subject being mentally stressed, mentally fatigued and/or cognitively challenged.

28. The use of claim 27, wherein the extract or medicament is administered to the subject prior to the subject being mentally stressed, mentally fatigued and/or cognitively challenged.

29. The use of claim 28, wherein the extract or medicament is administered at least or about 15 minutes prior to, at least or about 30 minutes prior to, at least or about 1 hour prior to, or at least or about 2 hours prior to, the subject being mentally stressed, mentally fatigued and/or cognitively challenged.

30. The use of any one of claims 18 to 26, wherein the subject is mentally stressed, mentally fatigued and/or cognitively challenged as a result of undergoing a test, examination or some other activity involving cognition.

31. The use of claim 30, wherein the extract or medicament is administered prior to commencement of the test, examination or other activity involving cognition.

32. The use of claim 31, wherein the extract or medicament is administered at least or about 15 minutes prior to, at least or about 30 minutes prior to, at least or about 1 hour prior to, or at least or about 2 hours prior to, commencement of the test, examination or other activity involving cognition.

33. The use of any one of claims 18 to 32, wherein the extract or medicament is administered in an amount of extract of between about 200 mg and about 2.0 g, or in an amount of extract of between about 300 mg and about 1.0 g, or in an amount of extract of between about 320 mg and about 960 mg, or in an amount of extract of between about 320 mg and about 640 mg.

34. The use of any one of claims 18 to 33, wherein the extract or medicament is administered in an amount of extract of at least about 320 mg, or in an amount of extract of at least about 640 mg, or in an amount of extract of at least about 960 mg.

35. The use of any one of claims 18 to 34, wherein the extract or medicament is administered orally.
